(19) United States
(12) Patent Application Publication
(10) Pub. No.: US 2012/0123503 A1
(43) Pub. Date: May 17, 2012

(54) PATIENT PROGRAMMER WITH CUSTOMIZABLE PROGRAMMING

(75) Inventor: William C. Phillips, Brooklyn Park, MN (US)

(73) Assignee: Medtronic, Inc., Minneapolis, MN (US)

(21) Appl. No.: 12/946,225

(22) Filed: Nov. 15, 2010

(51) Int. Cl. A61N 1/08

(52) U.S. Cl. 607/60

(57) ABSTRACT

This disclosure describes techniques for configuring external programmers for use with implantable medical devices. The techniques may include customizing a generic external programmer, e.g., a patient programmer, with an application for interfacing with the implanted medical device. In one example, a method includes receiving, from a clinician programmer, a selected one of a plurality of applications, wherein each of the applications is configured to control the operation of a patient programmer, the selected application is associated with a type of the IMD, and others of the plurality of applications are associated with different types of IMDs, and configuring, based on the received application, the patient programmer to present an interface that enables a user to interact with the IMD via the patient programmer.

### TECHNICAL FIELD

The disclosure relates to medical devices and, more particularly, to handheld programmers for medical devices.

### BACKGROUND

Medical devices are used to deliver therapy to patients to treat a variety of symptoms or conditions, and may be implantable or external. An implantable neurostimulator, for example, may treat symptoms or conditions such as chronic pain, tremor, Parkinson's disease, epilepsy, incontinence, or gastroparesis. The implantable medical device delivers neurostimulation therapy via one or more leads that include electrodes located proximate to the spinal cord, pelvic nerves, or stomach, or within the brain of a patient. In general, the implantable medical device delivers neurostimulation therapy in the form of electrical pulses.

A clinician selects values for a number of programmable parameters in order to define the neurostimulation therapy to be delivered to a patient. For example, the clinician may select an amplitude, which may be a current or voltage amplitude, and pulse width for a stimulation waveform to be delivered to the patient, as well as a rate at which the pulses are to be delivered to the patient. In addition, the clinician also selects particular electrodes within an electrode set to be used to deliver the pulses, and the polarities of the selected electrodes.

The clinician uses a clinician programmer to program the parameters into the implantable medical device. The implantable medical device may store multiple programs, however, which may be selected by the patient using a patient programmer. The patient may select different programs to modify therapy delivered by the implantable medical device, e.g., to achieve greater pain relief. Different programs may be appropriate for different times of day or different physical activities or postures.

The patient programmer communicates with the implantable medical device to modify programs using telemetry. The patient programmer is typically designed as a mobile device that may be carried by the patient throughout the course of the day. For example, the patient programmer may be a handheld device, and typically is powered by batteries within the device.

### SUMMARY

In general, this disclosure describes techniques for configuring external programmers for use with implantable medical devices. The techniques may include customizing a generic external programmer, e.g., a patient programmer, with an application that enables a user to interact with an implantable medical device (IMD) via the external programmer. In some cases, the application is selected from one or more applications stored on a clinician programmer. Each of the applications is associated with a type of IMD and is configured to the control the operation of a patient programmer. Once the application is selected, the application is transmitted from the clinician programmer to the generic external programmer, e.g., patient programmer. The application configures the generic external programmer to present an interface to the user that is appropriate for the type of IMD with which the external programmer will be used and enables a user to interact with the IMD via the patient programmer.

In one example, the disclosure is directed to a method comprising receiving, via a clinician programmer, user input that selects one of a plurality of applications, wherein each of the applications is configured to control the operation of a patient programmer, the selected application is associated with a type of the IMD, and others of the plurality of applications are associated with different types of IMDs, and transmitting, via the clinician programmer, the selected application to the patient programmer, wherein the patient programmer is configured to receive and implement the selected application to enable a user to interact with the IMD via the patient programmer.

In another example, the disclosure is directed to a system comprising a user interface, a memory device configured to store a plurality of applications, wherein each of the applications is configured to the control the operation of a patient programmer, and a processor. The processor is configured to receive user input that selects one of the plurality of applications at the time of initial programming of the implantable medical device (IMD), wherein the selected application is associated with a type of the IMD, and others of the plurality of applications are associated with different types of IMDs, and control transmission of the selected application to the patient programmer, wherein the patient programmer is configured to receive and implement the selected application to enable a user to interact with the IMD via the patient programmer.

In another example, the disclosure is directed to a computer-readable storage medium comprising instructions that, when executed by a processor, cause the processor to receive user input that selects one of a plurality of applications at the time of initial programming of the implantable medical device (IMD), wherein the selected application is associated with a type of the IMD, and others of the plurality of applications are associated with different types of IMDs, and control transmission of the selected application to the patient programmer, wherein the patient programmer is configured to receive and implement the selected application to enable a user to interact with the IMD via the patient programmer.

In another example, the disclosure is directed to a method comprising receiving, from a clinician programmer, a selected one of a plurality of applications, wherein each of the applications is configured to control the operation of a patient programmer, the selected application is associated with a type of the IMD, and others of the plurality of applications are associated with different types of IMDs, and configuring, based on the received application, the patient programmer to present an interface that enables a user to interact with the IMD via the patient programmer.

In another example, the disclosure is directed to a system comprising a user interface, a memory device configured to store an application, wherein the application is configured to control the operation of a patient programmer, and a processor. The processor is configured to receive, from a clinician programmer, a selected one of a plurality of applications for interfacing with an implantable medical device (IMD), wherein the selected application is associated with a type of IMD, and others of the plurality of applications are associated with different types of IMDs, and configure, based on the received application, the user interface to present an interface that enables a user to interact with the IMD via the patient programmer.

In another example, the disclosure is directed to a computer-readable storage medium comprising instructions that, when executed by a processor, cause the processor to receive, from a clinician programmer, a selected one of a plurality of applications for interfacing with an implantable medical device (IMD), wherein the selected application is associated with a type of IMD, and others of the plurality of applications are associated with different types of IMDs, and configure, based on the received application, a user interface to present an interface that enables a user to interact with the IMD via the patient programmer.

In another example, the disclosure is directed to a method comprising receiving a selected one of a plurality of applications, wherein each of the applications is configured to control the operation of a patient programmer, the selected application is associated with a type of the IMD, and others of the plurality of applications are associated with different types of IMDs, and assigning, based on the received application, a function to at least one input of the patient programmer.

The details of one or more aspects of the disclosure are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example therapy system that includes an implantable stimulator coupled to at least one stimulation lead.

FIG. 2 is a conceptual diagram illustrating another example therapy system that includes an implantable stimulator coupled to at least one stimulation lead.

FIG. 3 is a perspective view illustrating a medical device system including a clinician programmer, patient programmer and implantable medical device that may implement certain techniques of this disclosure.

FIG. 4 is a functional block diagram illustrating an example clinician programmer and an example patient programmer that may implement certain techniques of this disclosure.

FIG. 5 is a perspective view of an example patient programmer that may implement certain techniques of this disclosure.

FIG. 6 is a flow diagram illustrating an example method for configuring a generic external programmer for use with implantable medical devices using certain techniques of this disclosure.

FIG. 7 is a flow diagram illustrating another example method for configuring a generic external programmer for use with implantable medical devices using certain techniques of this disclosure.

FIG. 8 is a flow diagram illustrating another example method for configuring a generic external programmer for use with implantable medical devices using certain techniques of this disclosure.

### DETAILED DESCRIPTION

This disclosure describes techniques that allow a generic external programmer for use with an implantable medical device (IMD) to be programmed with an application for operating the patient programmer that is specific to a type of the IMD. The application allows the patient programmer to interface with the IMD, and cause the patient programmer to present an interface that allows a user of the patient programmer to program, e.g., modify, operational parameters of the IMD. At the time of implant or during the initial programming of the IMD in the patient, a clinician, for example, may select an application from one or more applications stored on a clinician programmer. For example, the clinician programmer may include one application for use with a first type of IMD configured to deliver therapeutic electrical stimulation to a patient for pain relief, another application for use with a second type of IMD configured to deliver one or more therapeutic agents via a drug pump, another application for use with a third type of IMD configured to provide deep brain stimulation, and another application for use with a fourth type of IMD configured to deliver therapeutic electrical stimulation to treat pelvic nerve disorders. A user may select one or more applications that are appropriate for the type of IMD implanted in the patient from amongst the applications stored on the clinician programmer.

Once the user selects an application, e.g., an application for use with an IMD configured to deliver therapeutic electrical stimulation to a patient for pain relief, the selected application is transmitted from the clinician programmer to the generic external programmer, e.g., patient programmer. The selected application then configures the generic external programmer to present an interface to the user that is appropriate for the IMD with which the programmer will be used. In this particular example, the selected application configures the generic patient programmer to present an interface to the patient that will allow the patient to, for example, adjust stimulation parameters such as delivery cycle, duration, voltage or current amplitude, pulse width, and pulse rate, or select from stored programs comprising electrical stimulation therapy parameter sets.

If the user had selected an application to be used with an IMD that delivers one or more therapeutic agents via a drug pump, for example, the selected application would configure the generic external programmer to present an interface to the user that is appropriate for the IMD that delivers drug therapy. In such an example, the selected application configures the generic patient programmer to present an interface to the patient that will allow the patient to, for example, specify the bolus size of a therapeutic agent that is delivered to a target tissue site, the frequency of bolus delivery, the concentration of the therapeutic agent delivered in each bolus, and the type of therapeutic agent delivered if the IMD is configured to deliver more than one type of therapeutic agent.

The techniques described in this disclosure permit the manufacture of generic external programmers that can be programmed at the time of implant of an IMD, or during the initial programming of the IMD in the patient, with an application. In addition, the techniques described herein allow an external programmer previously programmed with a first application to be reprogrammed, or re-imaged, with a second application that is different than the first application. That is, an application is not loaded onto a programmer during the manufacturing process, but is instead loaded at a clinic, for example. Allowing such generic external programmers to be manufactured may provide several advantages. For example, the manufacturing process may be simplified because only a single model of external programmer, which can later be configured via an application, needs to be manufactured.

In addition, a generic external programmer, which can later be configured via an application, may simplify supply chain management and inventory control by reducing the materials and the number of components required to build an external programmer. Multiple versions of external programmers may require different housings, hardware buttons, covers, and the like, which may increase the complexity of controlling inventory and supplying materials. Producing a single, generic external programmer, however, may reduce the number of material suppliers and the complexity of inventory control because only a single set of components for the generic external programmer is required to be sourced and stored.

Further, eliminating multiple versions of external programmers may reduce the opportunity for error in a manufacturing facility because components from one programmer version cannot be mistakenly assembled into another programmer version. In addition, if one or more applications are updated or introduced, the manufacturing process is not affected because the new or updated applications may be downloaded from a network onto the clinician programmer by a user at the time of implantation, for example.

Further still, a generic external programmer, which can be configured later via an application, may simplify distribution. Rather than carry multiple versions of external programmers, sales representatives, for example, need only carry a stockpile of generic external programmers and do not need to manage an inventory of different programmer versions.

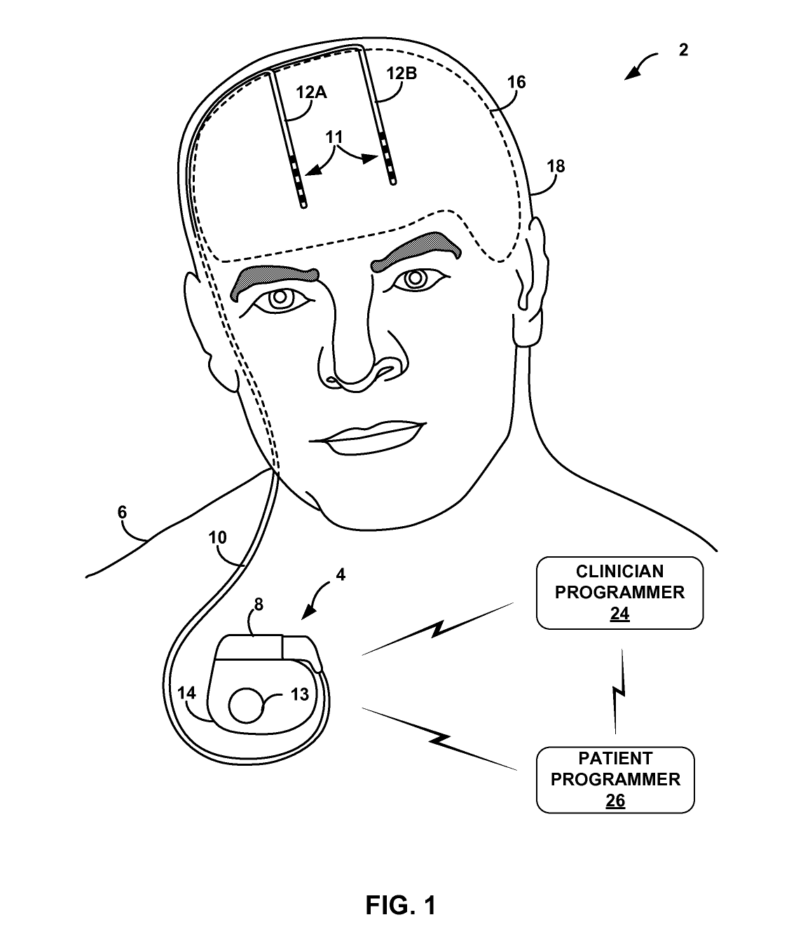

FIG. 1 is a conceptual diagram illustrating an example system 2 that may be used to deliver stimulation therapy to patient 6. Patient 6 ordinarily, but not necessarily, will be a human. Generally, therapy system 2 includes IMD 4 that delivers electrical stimulation to patient 6 via one or more implantable electrodes (not shown). The implantable electrodes may be deployed on one or more implantable medical leads, such as implantable medical lead 10, and in some cases on a can electrode. The electrical stimulation may be in the form of constant current or voltage pulses or substantially continuous waveforms. Various parameters of the pulses or waveforms may be defined by a stimulation program. The pulses or waveforms may be delivered substantially continuously or in bursts, segments, or patterns, and may be delivered alone or in combination with pulses or waveforms defined by one or more other stimulation programs. One or more of the electrodes may be located on a housing 14, i.e., “can” or “case,” of the IMD 4. In addition, in some cases, implantable electrodes may be deployed on a leadless stimulator.

In the example illustrated in FIG. 1, IMD 4 is implanted within a subcutaneous pocket in a clavicle region of patient 6. Stimulator 4 generates programmable electrical stimulation, e.g., a current waveform or current pulses, and delivers the stimulation via an implantable medical lead 10 carrying an array of implantable stimulation electrodes 11. In some cases, multiple implantable leads may be provided. In the example of FIG. 1, a distal end of lead 10 is bifurcated and includes two lead segments 12A and 12B (collectively “lead segments 12”). Lead segments 12A and 12B each include a set of electrodes forming part of the array of electrodes 11. In various examples, lead segments 12A and 12B may each carry four, eight, or sixteen electrodes. In FIG. 1, each lead segment 12A, 12B carries four electrodes, configured as ring electrodes at different axial positions near the distal ends of the lead segments. Throughout the remainder of this disclosure, for purposes of simplicity, the disclosure may generally refer to electrodes carried on “leads” rather than “lead segments.”

FIG. 1 further depicts a housing, or can, electrode 13 carried by housing 14. Housing electrode 13 may be formed integrally with an outer surface of hermetically-sealed housing 14 of IMD 4, or otherwise coupled to housing 14.

In some examples, lead 10 may also carry one or more sense electrodes to permit IMD 4 to sense electrical signals from patient 6. Some of the stimulation electrodes may be coupled to function as stimulation electrodes and sense electrodes on a selective basis. In other examples, IMD 4 may be coupled to one or more leads which may or may not be bifurcated. In such examples, the leads may be coupled to IMD 4 via a common lead extension or via separate lead extensions.

A proximal end of lead 10 may be electrically and mechanically coupled to header 8 on IMD 4 either directly or indirectly via a lead extension. Conductors in the lead body may electrically connect stimulation electrodes located on lead segments 12 to IMD 4. Lead 10 traverses from the implant site of IMD 4 along the neck of patient 6 to cranium 18 of patient 6 to access brain 16. Lead segments 12A and 12B are implanted within the right and left hemispheres, respectively, in order to deliver electrical stimulation to one or more regions of brain 16, which may be selected based on the patient condition or disorder.

IMD 4 may deliver, for example, deep brain stimulation (DBS) or cortical stimulation (CS) therapy to patient 6 via the electrodes carried by, i.e., located on, lead segments 12 to treat any of a variety of neurological disorders or diseases. Example neurological disorders may include depression, dementia, obsessive-compulsive disorder and movement disorders, such as Parkinson's disease, spasticity, epilepsy, and dystonia. DBS also may be useful for treating other patient conditions, such as migraines and obesity. However, the disclosure is not limited to the delivery of DBS or CS therapy.

Lead segments 12A, 12B may be implanted within a desired location of brain 16 through respective holes in cranium 18. Lead segments 12A, 12B may be placed at any location within brain 16 such that the electrodes located on lead segments 12A, 12B are capable of providing electrical stimulation to targeted tissue during treatment. Example locations for lead segments 12A, 12B within brain 16 may include the pedunculopontine nucleus (PPN), thalamus, basal ganglia structures (e.g., globus pallidus, substantia nigra, subthalmic nucleus), zona inserta, fiber tracts, lenticular fasciculus (and branches thereof), ansa lenticularis, and/or the Field of Forel (thalamic fasciculus). In the case of migraines, lead segments 12 may be implanted to provide stimulation to the visual cortex of brain 16 in order to reduce or eliminate migraine headaches afflicting patient 6. However, the target therapy delivery site may depend upon the patient condition or disorder being treated.

Therapy system 2 also may include a clinician programmer 24 and/or a patient programmer 26. Clinician programmer 24 may be a handheld computing device that permits a clinician to program stimulation therapy for patient 6 via a user interface, e.g., using input keys and a display or a touch screen display. For example, using clinician programmer 24, the clinician may specify stimulation parameters, i.e., create programs, for use in delivery of stimulation therapy. Clinician programmer 24 may support telemetry (e.g., radio frequency (RF) telemetry) with IMD 4 to download programs and, optionally, upload operational or physiological data stored by IMD 4. In this manner, the clinician may periodically interrogate IMD 4 to evaluate efficacy and, if necessary, modify the programs or create new programs. As described in more detail below, using the techniques of this disclosure, a user may also interact with clinician programmer 24 to program IMD 4, e.g., select values for operational parameters of the IMD, during the initial implantation of IMD 4 within patient 6 or during subsequent in-clinic visits.

Like clinician programmer 24, patient programmer 26 may be a handheld computing device. Patient programmer 26 may also include a display and input keys to allow patient 6 to interact with patient programmer 26 and IMD 4. In this manner, patient programmer 26 provides patient 6 with a user interface for control of the stimulation therapy delivered by IMD 4. For example, patient 6 may use patient programmer 26 to start, stop or adjust electrical stimulation therapy. In particular, patient programmer 26 may permit patient 6 to adjust stimulation parameters of a program such as duration, current or voltage amplitude, pulse width and pulse rate. Patient 6 may also select a program, e.g., from among a plurality of stored programs, as the present program to control delivery of stimulation by IMD 4.

In some examples, IMD 4 delivers stimulation according to a group of programs at a given time. Each program of such a program group may include respective values for each of a plurality of therapy parameters, such as respective values for each of current or voltage amplitude, pulse width, pulse shape, pulse rate and electrode configuration (e.g., electrode combination and polarity). IMD 4 may interleave pulses or other signals according to the different programs of a program group, e.g., cycle through the programs, to simultaneously treat different symptoms or different body regions, or provide a combined therapeutic effect. In such examples, clinician programmer 24 may be used to create programs, and assemble the programs into program groups. Patient programmer 26 may be used to adjust stimulation parameters of one or more programs of a program group, and select a program group, e.g., from among a plurality of stored program groups, as the current program group to control delivery of stimulation by IMD 4.

IMD 4, clinician programmer 24, and patient programmer 26 may communicate via cables or a wireless communication, as shown in FIG. 1. Clinician programmer 24 and patient programmer 26 may, for example, communicate via wireless communication with IMD 4 using RF telemetry techniques known in the art. Clinician programmer 24 and patient programmer 26 also may communicate with each other using any of a variety of local wireless communication techniques, such as RF communication according to the 802.11 or Bluetooth specification sets, infrared communication, e.g., according to the IrDA standard, or other standard or proprietary telemetry protocols. Each of clinician programmer 24 and patient programmer 26 may include a transceiver to permit bi-directional communication with IMD 4.

As described in more detail below, using the techniques of this disclosure, a user may select one application from among a plurality of applications stored on clinician programmer 24. Each of the applications is configured to the control the operation of a patient programmer. A selected application is associated with a type of IMD, e.g., a first type of IMD may be configured to deliver therapeutic electrical stimulation to a patient for pain relief, and others of the plurality of applications are associated with different types of IMDs, e.g., a second type of IMD may be configured to deliver one or more therapeutic agents via a drug pump, a third type of IMD may be configured to provide deep brain stimulation, fourth type of IMD may be configured to deliver therapeutic electrical stimulation to treat pelvic nerve disorders, and the like. Once the user selects an application, e.g., an application for use with a first type of IMD configured to deliver electrical stimulation to a patient in the form of DBS, the selected application is transmitted from clinician programmer 24 to the generic external programmer, e.g., patient programmer 26. The selected application then configures patient programmer 26 to present an interface to the user that is appropriate for IMD 4. In this particular example, the selected application configures the generic patient programmer to present an interface to the patient that will allow the patient to, for example, adjust stimulation parameters such as delivery cycle, duration, voltage or current amplitude, pulse width, and pulse rate, or select from stored programs comprising electrical stimulation therapy parameter sets. In other words, the selected application enables a user to interact with the IMD via the patient programmer.

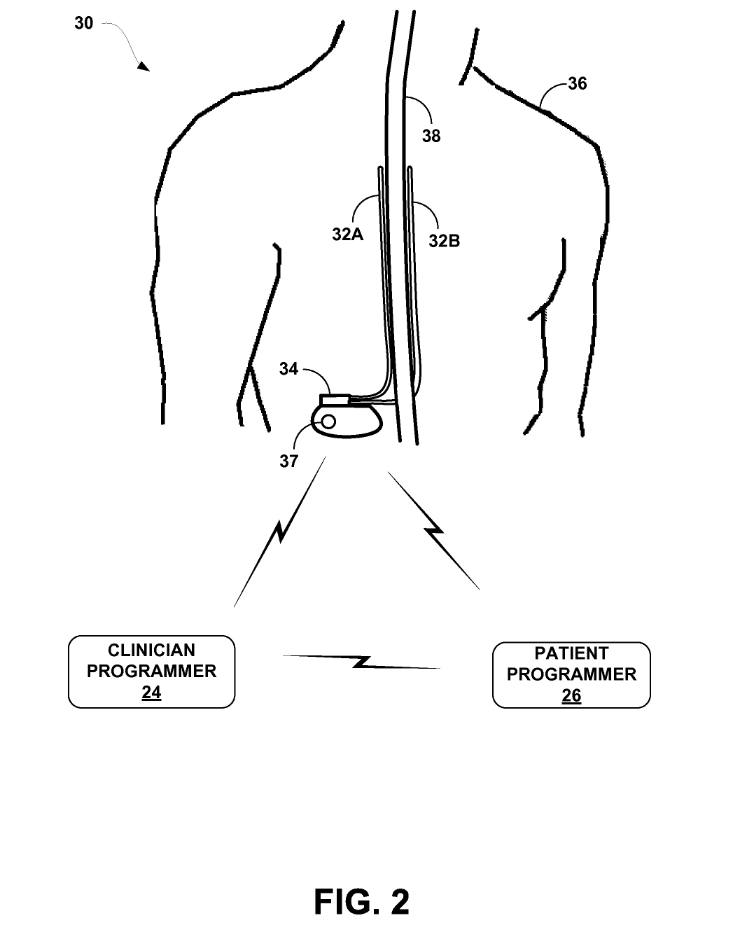

FIG. 2 is a conceptual diagram illustrating system 30 that delivers stimulation therapy to spinal cord 38 of patient 36. FIG. 2 represents another example of a therapy delivery system that may implement various techniques of this disclosure. In particular, FIG. 2 represents an electrical stimulation system that may allow a generic external programmer, e.g., patient programmer 26, for use with an implantable medical device, e.g., IMD 34, to be programmed at the time of implant with an application to interface with IMD 34. Other electrical stimulation systems that may implement various techniques of this disclosure may be configured to deliver electrical stimulation to gastrointestinal organs, pelvic nerves or muscle, peripheral nerves, or other stimulation sites. In addition, drug delivery systems may implement techniques of this disclosure. Further, the techniques of this disclosure are not limited to therapy delivery systems but may also be used in conjunction with non-therapy systems, e.g., systems that include one or more IMDs that monitor one or more biological parameters. In the example of FIG. 2, system 30 delivers stimulation therapy from IMD 34 to spinal cord 38 via one or more electrodes (not shown) carried by, i.e., located on, implantable medical leads 32A and 32B (collectively “leads 32”) as well as the housing of IMD 34, e.g., housing electrode 37. System 30 and, more particularly, IMD 34 may operate in a manner similar to IMD 4 (FIG. 1).

In the example of FIG. 2, the distal ends of leads 32 carry electrodes that are placed adjacent to the target tissue of spinal cord 38. The proximal ends of leads 32 may be both electrically and mechanically coupled to implantable stimulator 4 either directly or indirectly via a lead extension and header. Alternatively, in some examples, leads 32 may be implanted and coupled to an external stimulator, e.g., through a percutaneous port. In additional example implementations, stimulator 34 may be a leadless stimulator with one or more arrays of electrodes arranged on a housing of the stimulator rather than leads that extend from the housing.

IMD 34 may be implanted in patient 36 at a location minimally noticeable to the patient. For SCS, stimulator 34 may be located in the lower abdomen, lower back, or other location to secure the stimulator. Leads 32 are tunneled from stimulator 34 through tissue to reach the target tissue adjacent to spinal cord 38 for stimulation delivery. At the distal ends of leads 32 are one or more electrodes (not shown) that transfer the stimulation pulses from the lead to the tissue substantially simultaneously with stimulation pulses delivered via a housing electrode, e.g., electrode 37.

IMD 34 delivers stimulation to spinal cord 38 to reduce the amount of pain perceived by patient 36. As mentioned above, however, the stimulator may be used with a variety of different therapies, such as peripheral nerve stimulation (PNS), peripheral nerve field stimulation (PNFS), deep brain stimulation (DBS), cortical stimulation (CS), pelvic floor stimulation, peripheral nerve stimulation, gastric stimulation, and the like. The stimulation delivered by IMD 34 may take the form of stimulation pulses or continuous stimulation waveforms, and may be characterized by controlled current or voltage levels, as well as programmed pulse widths and pulse rates in the case of stimulation current pulses. Stimulation may be delivered via selected combinations of electrodes located on one or both of leads 32 and on the housing. Stimulation of spinal cord 38 may, for example, prevent pain signals from traveling through the spinal cord and to the brain of the patient. Patient 34 perceives the interruption of pain signals as a reduction in pain and, therefore, efficacious therapy.

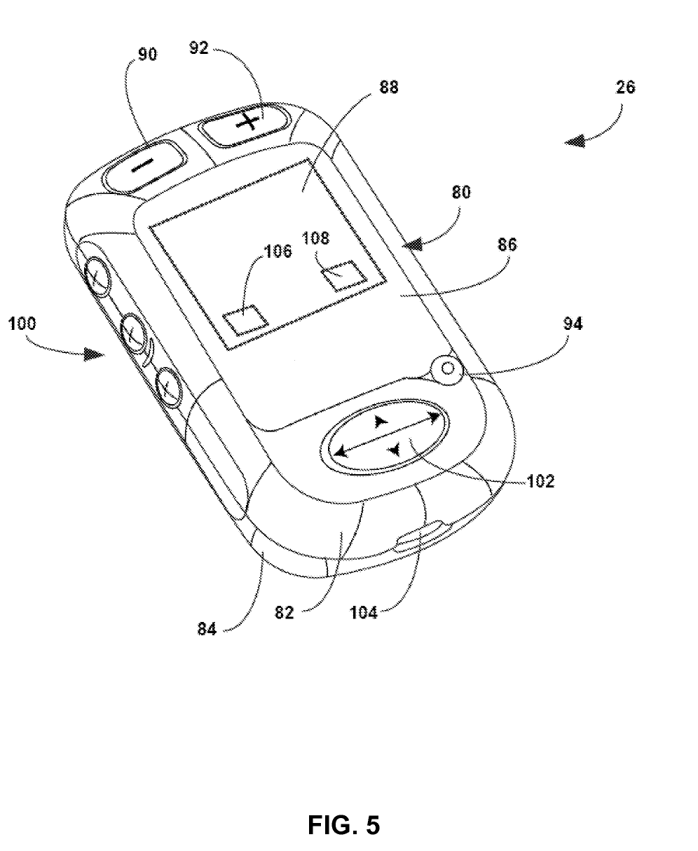

With reference to FIG. 2, a user, such as a clinician or patient 36, may interact with a user interface of clinician programmer 24 to program IMD 34. Programming of IMD 34 may refer generally to the generation and transfer of commands, programs, or other information to control the operation of the stimulator. For example, clinician programmer 24 may transmit programs, parameter adjustments, program selections, group selections, or other information to control the operation of IMD 34, e.g., by wireless telemetry. In addition, in accordance with certain aspects of this disclosure, clinician programmer 24, a general-purpose personal computer (PC) (e.g., a desktop PC, a laptop PC, and the like), a tablet PC, a network server, or some other device via a wired or wireless connection may transmit parameters to patient programmer 26 that assign, or map, functions to the input mechanisms, e.g., buttons, scroll wheels, soft keys, and the like, of patient programmer 26, as is described in more detail below with respect to FIG. 5. Patient programmer 26 may support adjustment and selection of such programs by a patient during ordinary use. Communication between clinician programmer 24, patient programmer 26, and IMD 34 is similar to that described above with respect to FIG. 1 and, for purposes of conciseness, will not be described again.

Clinician programmers 24 of FIGS. 1 and 2 may be substantially similar. Indeed, clinician programmers 24 of FIGS. 1 and 2 may be the same clinician programmer. In accordance with the techniques of this disclosure, a user may select an application from among a plurality of applications stored on clinician programmer 24. For example, clinician programmer 24 of FIG. 2 may include an application that controls IMD 4 of FIG. 1, e.g., a first type of IMD, to deliver deep brain stimulation as well as an application that controls IMD 34 of FIG. 2, a second type of IMD, to deliver stimulation to the spinal cord to reduce the amount of pain perceived by patient 36. Once a user selects an application, e.g., a application for use with an IMD configured to deliver stimulation to the spinal cord to reduce the amount of perceived pain, the selected application is downloaded from clinician programmer 24 to the generic external programmer, e.g., patient programmer 26. The selected application then configures the patient programmer 26 to present an interface to the user that is appropriate for IMD 34. In this particular example, the selected application configures the generic patient programmer to map functions to programmer input mechanisms (e.g., buttons, scrolls wheels, etc.) and present an interface to the patient that will allow the patient to, for example, adjust stimulation parameters such as delivery cycle, duration, voltage or current amplitude, pulse width, and pulse rate, or select from stored programs comprising electrical stimulation therapy parameter sets. In other words, the selected application enables a user to interact with the IMD via the patient programmer and to interact with the programmer itself (e.g., provide commands to retrieve information, etc.)

FIGS. 1 and 2 depict implantable medical devices that may deliver electrical stimulation. However, the techniques of this disclosure are not limited to systems that include implantable medical devices that deliver electrical stimulation. Rather, although not depicted, the techniques of this disclosure also support, for example, implantable medical devices that include a drug delivery system configured to deliver a therapeutic agent in some metered or other desired flow dosage to a therapy site within a patient. In such example configurations, patient programmer 26 provides a patient with an interface to control delivery of a therapeutic agent. For example, the patient may use patient programmer 26 to start, stop, or adjust delivery of a therapeutic agent. Patient programmer 26 may control the operation of a drug pump housed within the IMD with the aid of instructions that are stored in a memory. For example, the instructions may define therapy programs that specify the bolus size of a therapeutic agent that is delivered to a target tissue site within the patient from a reservoir via a catheter. The therapy programs may also include other therapy parameters, such as the frequency of bolus delivery, the concentration of the therapeutic agent delivered in each bolus, the type of therapeutic agent delivered (if the IMD is configured to deliver more than one type of therapeutic agent), and so forth.

Further, as indicated above, the techniques of this disclosure are not limited to systems that deliver therapy. Rather, the techniques described herein may be used in conjunction with implantable medical devices, e.g., sensors, that monitor certain biological parameters. Patient programmer 26 may configure the operation of sensors, e.g., sensitivity settings, and adjust or provide algorithms for detecting patient conditions or events.

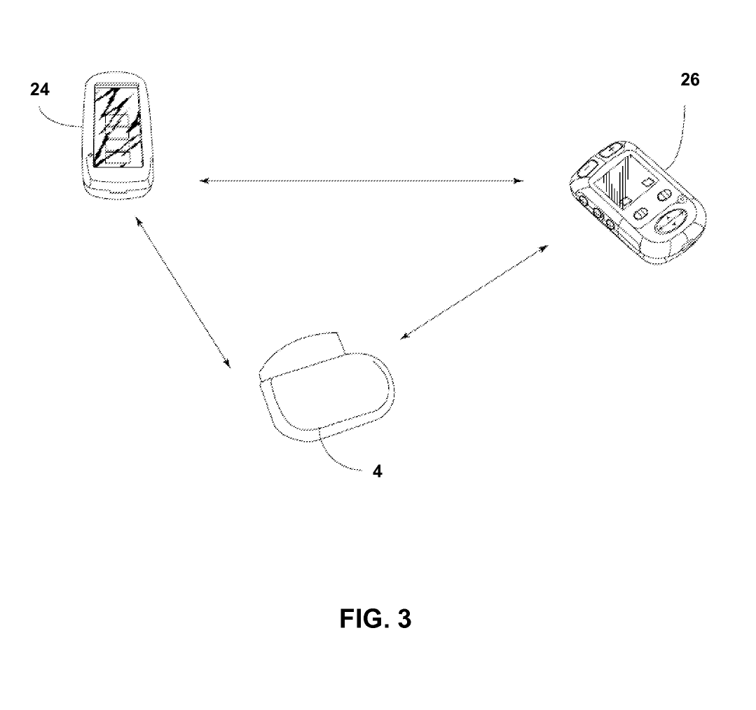

FIG. 3 is a perspective view illustrating a therapy delivery system including a clinician programmer 24, patient programmer 26 and IMD 4 (or IMD 34 of FIG. 2). The system includes IMD 4, which delivers therapy, e.g., therapeutic electrical stimulation or therapeutic agents, to patient 6. Clinician programmer 24 is used by a clinician to program therapy for patient 6. In particular, the clinician may use programmer 24 to create therapy programs. For example, as part of the program creation process to create therapeutic electrical stimulation programs, programmer 24 may allow the clinician to identify parameter settings and electrode configurations that enable IMD 4 to deliver neurostimulation therapy that is desirable in terms of, for example, symptom relief, coverage area relative to symptom area, and side effects. As another example, as part of the program creation process to create programs that control delivery of therapeutic agents, programmer 24 may allow the clinician to identify parameter settings that enable IMD 4 to titrate delivery of one or more therapeutic agents to patient 6.

Clinician programmer 24 may also allow the clinician to identify parameter settings that enable IMD 4 to deliver effective therapy with desirable device performance characteristics, e.g., low battery consumption. Clinician programmer 24 may control IMD 4 to test parameter settings in order to allow a clinician to identify desirable configurations in an efficient manner.

Using the techniques of this disclosure, clinician programmer 24 may be configured to store one or more applications. Once clinician programmer 24 has loaded IMD 4 with one or more therapy programs, a user, e.g., a clinician or sales representative, may select one of the applications stored on clinician programmer 24. Clinician programmer 24 may then transmit, e.g., via a wired connection, a wireless connection, or via a computer-readable media (such as a memory stick), the selected application to a generic external programmer, namely patient programmer 26. Once downloaded, the selected application configures the generic patient programmer 26 to map programmer functions to buttons, scroll wheels, and the like, and to present an interface to the user that is appropriate for the IMD with which the external programmer will be used. For example, the interface for an IMD that is configured to deliver a therapeutic agent in some metered or other desired flow dosage to a therapy site within a patient may be different from the interface for an IMD that is configured to deliver DBS, as in FIG. 1, or pain relief, as in FIG. 2. Once patient programmer 26 is configured by the selected application, the patient may use patient programmer 26 to program operational parameters of the IMD, e.g., modify and select programs and parameter settings for therapy delivery.

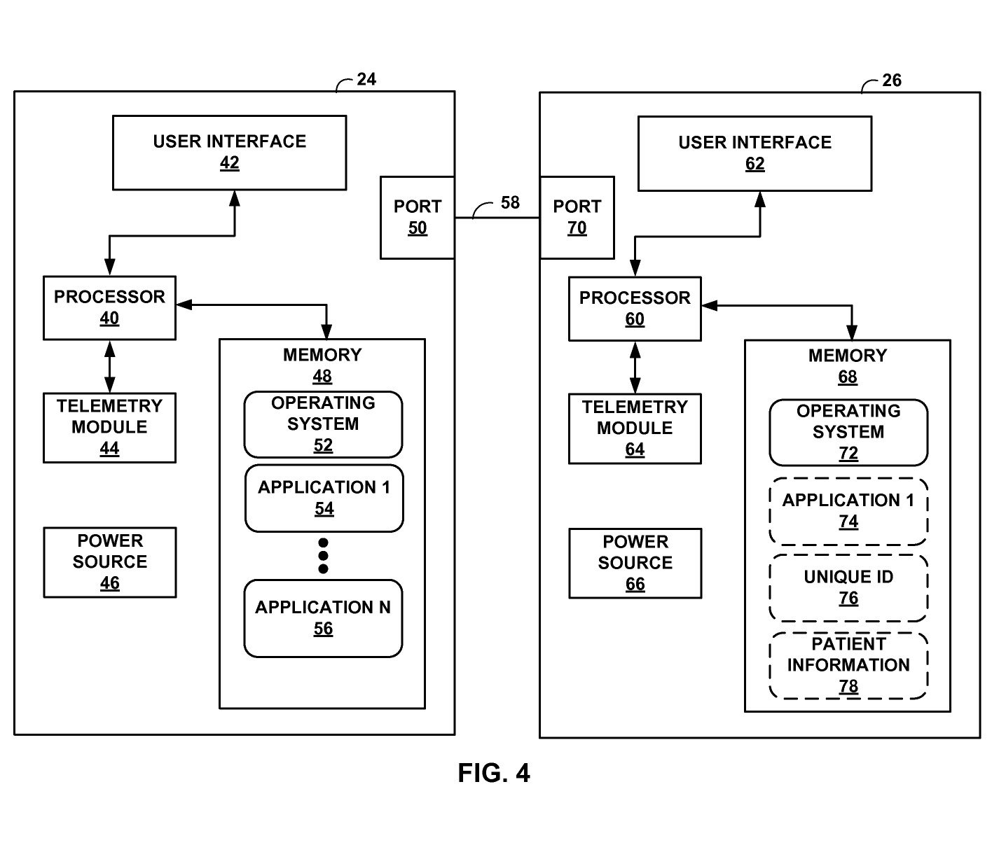

FIG. 4 is a functional block diagram illustrating example components of a clinician programmer and a patient programmer that may implement the techniques of this disclosure. Clinician programmer 24 may include processor 40, user interface 42, telemetry module 44, power source 46, memory 48, and one or more ports 50. In general, processor 40 controls user interface 42, stores and retrieves data to and from memory 48, and controls transmission of data with an IMD through telemetry module 44 and with a patient programmer through a wired connection from port 50 or through a wireless connection via telemetry module 44.

User interface 42 may include a screen and one or more input buttons that allow clinician programmer 24 to receive input from a user, e.g., a clinician or sales representative. The screen may be a liquid crystal display (LCD), plasma display, dot matrix display, or touch screen. The input buttons may include a touch pad, increase and decrease buttons, emergency shut off button, and other input media needed to control the stimulation therapy. In accordance with the techniques of this disclosure, user interface 42 is configured to receive, at the time of initial programming of an IMD or at the time of implant, user input that selects one of the applications 54, 56 stored in memory 48.

Memory 48, a computer-readable storage medium, may store instructions that cause processor 40 to provide various aspects of the functionality ascribed to clinician programmer 24 in this disclosure. Memory 48 may include any fixed or removable magnetic, optical, or electrical media, such as RAM, ROM, CD-ROM, magnetic disks, EEPROM, or the like. Memory 48 may also include a removable memory portion that may be used to provide memory updates or increases in memory capacities. A removable memory may also allow patient data to be easily transferred to another computing device, or to be removed before programmer 24 is used to program therapy for another patient. Memory 48 may also store information that controls operation of an IMD, such as therapy delivery values.

Consistent with the techniques of this disclosure, memory 48 may store operating system 52 and any number of applications, shown as application 1 (54) through application N (56) in the illustrated example. Each application is a software application that, when installed on generic patient programmer 26, configures the generic programmer to present an interface to the user that is appropriate for the IMD with which the patient programmer will be used. For example, if a patient is fitted with an IMD for pain relief, as in FIG. 2, application 1 (54) may configure patient programmer 26 and, in particular, user interface 62, to display the leads implanted within patient 36, the electrodes on the leads, as well as control inputs that allow patient 36 to alter various electrical stimulation parameters such as delivery cycle, amplitude, width, or the like, thereby specifying the manner in which electrical stimulation should be delivered to the patient via the IMD. As another example, if a patient is fitted with an IMD for delivery of a therapeutic substance (not shown), application N (56) may configure patient programmer 26 and, in particular, user interface 62, to allow the patient to provide feedback regarding the efficacy of treatment as well as request a therapeutic bolus, for example.

Memory 48 may also store newly released applications that may interface with newly released implantable medical devices. For example, upon release of a new IMD, processor 40 may retrieve, e.g., from a website, flash drive or other computer-readable storage media, and the like, the new application that will interface with the new IMD. Then, processor 40 may transmit the new application to generic patient programmer 26, which will configure itself to present an interface to the user that is appropriate for the new IMD. In this manner, the techniques of this disclosure may remove manufacturing operations from software distribution for the patient programmer. That is, the manufacturer of the generic patient programmer does not need to know about the new IMD because software distribution for the patient programmer happens via clinician programmer 24 and not via the manufacturing process. Thus, the techniques of this disclosure may simplify new product introductions.

While clinician programmer 24 may be any type of computing device, the clinician programmer may preferably be a hand-held device with a display and input mechanism associated with user interface 42 to allow interaction between the clinician, for example, and clinician programmer 24. The clinician programmer may differ from patient programmer 26 by having additional features not offered to the patient for security, performance, or complexity reasons.

Processor 40 may include one or more processors such as a microprocessor, a controller, a digital signal processor (DSP), an application-specific integrated circuit (ASIC), a field programmable gate array (FPGA), discrete logic circuitry, or the like. Processor 40 may control user interface 42 to display information, and perform certain functions when requested by a user via input to the user interface. Processor 40 may retrieve data from and/or store data in memory 48 in order to perform the functions of clinician programmer 24 described in this disclosure. For example, upon receiving user input that selects one of the applications 54, 56 stored in memory 48 via user interface 42, processor 40 retrieves the selected application, e.g., application 1 (54), and prepares to transmit the selected application to patient programmer 26. The functions attributed to processor 40 in this disclosure may be embodied as software, firmware, hardware or any combination thereof.

Telemetry module 44 allows the transfer of data to and from patient programmer 26 and an IMD. To support RF communication, telemetry module 44 may include appropriate electronic components, such as amplifiers, filters, mixers, encoders, decoders, and the like. In some examples, processor 40 controls telemetry module 44 to transmit the selected application to patient programmer 26. In addition, processor 40 may control telemetry module 44 to exchange information with patient programmer 26 as well as an IMD. Further, processor 40 may control telemetry module 44 to transmit to patient programmer 26 a unique identifier 76 that identifies the IMD. In general, an IMD is programmed with a unique identifier. This unique identifier permits clinician programmer 24 or patient programmer 26 to specify and communicate with a particular IMD without affecting other nearby implantable medical devices. Once patient programmer 26 receives unique identifier 76, or key, patient programmer 26 is “bonded” to the IMD with that particular unique identifier. Thus, the unique identifier provides a level of security and safety that reduces or eliminates the ability of one patient's patient programmer to reprogram another patient's IMD.

In other examples, processor 40 transmits the selected application to patient programmer 26 from port 50 via a wired connection. For example, port 50 may be a Universal Serial Bus (USB) port, a Joint Test Action Group (JTAG) interface, conforming to IEEE 1149.1 boundary-scan standard, or some other interface that supports transmission of data over wired connection 58 between port 50 of clinician programmer 24 and port 70 of patient programmer 70.

In addition to a selected application and unique identifier, clinician programmer 24 may also transmit patient information 78 to patient programmer 26. For instance, clinician programmer 24 may transmit patient information 78 such as the patient's name, the date of implant, the reason(s) for the implant, and the name of the implanting physician to patient programmer 26 for storage in memory 68.

Power source 46 delivers operating power to the components of programmer 24. Power source 46 may be a rechargeable battery, such as a lithium ion or nickel metal hydride battery. Other rechargeable or conventional batteries may also be used. In some cases, clinician programmer 24 may be used when coupled to an alternating current (AC) outlet, i.e., AC line power, either directly or via an AC/DC adapter. Power source 46 may include circuitry to monitor power remaining within a battery. In this manner, user interface 42 may provide a current battery level indicator or low battery level indicator when the battery needs to be replaced or recharged. In some cases, power source 46 may be capable of estimating the remaining time of operation using the current battery.

Patient programmer 26 may include processor 60, user interface 62, telemetry module 64, power source 66, memory 68, and one or more ports 70. In general, processor 60 controls user interface 62, stores and retrieves data to and from memory 68, and controls transmission of data with an IMD through telemetry module 64 and with a clinician programmer through a wired connection from port 70 or through a wireless connection via telemetry module 64. The components of patient programmer 26 are similar to the components described above with respect to clinician programmer 24 and, for purposes of conciseness, will not be described in detail again. While patient programmer 24 may generally be described as a hand-held computing device, the patient programmer may be a notebook computer, a cell phone, a personal digital assistant, or a workstation, for example.

Memory 68, a computer-readable storage medium, may store instructions that cause processor 60 to provide various aspects of the functionality ascribed to patient programmer 26 in this disclosure. Memory 68 stores operating system 72, which may, in some examples, be the same as operating system 52 of clinician programmer 24. Operating system 72 is configured such that programmer 24 may receive and implement a user-selected application that is loaded by the clinician programmer, thereby allowing patient programmer 26 to interface with clinician programmer 24. In addition, operating system 72 allows generic patient programmer 24 to be powered up during manufacturing and functionally tested.

Again, clinician programmer 24 transmits an application to patient programmer 26 at the time of implant, during the initial programming of the IMD, or during a reprogramming of a patient programmer (e.g., if a patient receives a new IMD, then the patient programmer may be reprogrammed and reused). In this manner, the manufacturing process need not be aware of different applications and need only produce a generic external programmer to be programmed at a later time.

Consistent with the techniques of this disclosure, memory 68 may receive and store the application that was selected by a user from among the applications stored in memory 48 of clinician programmer 24 and transmitted from clinician programmer 24 to patient programmer 26. For instance, if a user selected application 1 from among the N applications stored in memory 48 of clinician programmer 24, then processor 60 of patient programmer 26 stores application 1 in memory 68, as shown at 74.

Once stored in memory 68, the transmitted application, e.g., application 1 (74) loads (either manually or automatically) and configures patient programmer 26 to present a user interface to the user that is appropriate for the IMD with which the patient programmer will be used. In other words, patient programmer 26 is configured, based on the received application, to present an interface to a user that may be used to control the IMD implanted within the patient. For example, if a patient is fitted with an IMD for pain relief, as in FIG. 2, application 1 (54) may configure patient programmer 26 to display the leads implanted within patient 36, the electrodes on the leads, as well as control inputs that allow patient 36 to alter various electrical stimulation parameters such as delivery cycle, amplitude, width, or the like, thereby specifying the manner in which electrical stimulation should be delivered to the patient via the IMD.

As mentioned above, clinician programmer 24 may transmit a unique identifier that identifies the IMD. Either prior to or after receiving the selected application, patient programmer 26 receives, via telemetry module 64 or port 70, the unique identifier that bonds patient programmer 26 to a specific IMD. Upon receiving the unique identifier, processor 60 of patient programmer 26 stores the unique identifier in memory 68, shown at 76.

In addition, using certain techniques of this disclosure, various hardware circuitry of patient programmer 26, e.g., telemetry module 64, may be configured based on the received application. For example, the received application may configure telemetry module 64 such that particular filters, e.g., bandpass filters, are selected to process received telemetry signals differently. In this manner, an application transmitted from clinician programmer 24 to patient programmer 26 may configure telemetry module 64 differently in order to allow patient programmer 26 to communicate with different types of IMDs.

For example, a first application transmitted from a clinician programmer to a first patient programmer that allows the patient programmer to communicate with an IMD that delivers electrical stimulation therapy may configure a telemetry module to use a first set of filters to process received telemetry signals, while a second application transmitted from the clinician programmer to a second patient programmer that allows the second patient programmer to communicate with an IMD that delivers therapeutic agents may configure a telemetry module to use a second set of filters to process received telemetry signals.

In a similar manner, in example implementations in which a patient programmer includes recharge circuitry to replenish a rechargeable power source of an implanted device, various parameters may be programmed to configure the recharge circuitry of the patient programmer to be compatible with a particular power source and control circuit of the IMD with which the patient programmer will be used.

The hardware circuitry configuration techniques described above are not limited to telemetry or recharge modules, however. Rather, other hardware circuitry may be configured based on the received application. For example, user interface 62 of patient programmer 26 may be configured using the received application. In particular, a display of user interface 62 may have its layout, icons, soft keys, color, and the like, configured based on the received application so that a patient programmer that will communicate with an IMD that delivers electrical stimulation therapy may be configured differently than a patient programmer that will communicate with an IMD that delivers a therapeutic agent, for example.

Memory 68 may include specific therapy programs (not shown) comprising electrical stimulation parameters or parameters related to the delivery of a therapeutic agent. For example, the programs may include data that defines the parameters of electrical pulses delivered to the patient in the case of therapeutic electrical stimulation. When a new therapy program is requested by the IMD or the patient, processor 60 retrieves one of the therapy programs from memory 68 and transmits the retrieved program to the IMD in order adjust the electrical stimulation. Alternatively, the patient may generate a new program during therapy and store it in memory 68. As indicated above, the therapy programs which include, for example, parameters for controlling delivery of therapeutic electrical stimulation or therapeutic drug delivery and different from the applications used to configure the external programmer.

Processor 60 may control telemetry module 44 to exchange information with patient programmer 26 as well as an IMD. Further, processor 40 may control telemetry module 44 to transmit to patient programmer 26 a unique identifier that identifies the IMD. In general, an IMD is programmer with a unique identifier. This unique identifier permits clinician programmer 24 or patient programmer 26 to specify and communicate with a particular IMD without affecting other nearby implantable medical devices. Once patient programmer 26 receives the unique identifier, or key, patient programmer 26 is “bonded” to the IMD with that particular unique identifier. Thus, the unique identifier provides a level of security and safety that reduces or eliminates the ability of a patient programmer to reprogram another patient's IMD.

As indicated above, in addition to a selected application and unique identifier, clinician programmer 24 may also transmit patient information to patient programmer 26. For instance, clinician programmer 24 may transmit patient information such as the patient's name, the date of implant, the reason(s) for the implant, and the name of the implanting physician to patient programmer 26 for storage in memory 68. Upon receiving the patient information, processor 60 stores the patient information in memory 68, shown at 78.

It should be noted that application 1 (74), unique identifier 76, and patient information 78 stored in memory 68 of generic patient programmer 26 are indicated with dashed-line outlines because they are specific to the type of IMD implanted in the patient and are not initially stored in memory 68, thereby permitting patient programmer 26 to remain generic.

User interface 62 may be designed to receive an indication from the patient to deliver electrical stimulation or a therapeutic bolus in the case of a drug delivery system. The indication may be in the form of a patient input such as pressing a button representing the start of therapy, or selecting an icon from a touch screen, for example. In alternative examples, user interface 62 may receive an audio cue from the patient, e.g., the patient speaks into a microphone in order to perform functions such as beginning stimulation therapy. Patient programmer 26 acts as an intermediary for patient to communicate with the IMD for the duration of therapy.

FIG. 5 is a perspective view of an example patient programmer that may implement certain techniques of this disclosure. For example, using certain techniques of this disclosure, clinician programmer 24 may transmit parameters that assign, or map, a function to each of one or more inputs of patient programmer 26, as described in more detail below. As shown in FIG. 5, patient programmer 26 includes a housing 80. Housing 80 may be formed of molded plastic and may include front housing cover 82 and bottom housing cover 84, as well faceplate 86 with a transparent display section 88. Faceplate 86 may be formed of a clear plastic material. Front cover 82 includes a number of buttons 90, 92, 94, 96, 98, 100, 102 that serve as input mechanisms, or inputs, to control various aspects of the functionality of patient programmer 26. Front cover 82 may include apertures that permit buttons 90, 92, 94, 96, 98, 100, 102 to protrude through the front cover from the interior of housing 80. Front cover 82 may also include an infrared interface window 104 that exposes an infrared (e.g., IRDA) transmitter and receiver, for example.

Buttons 90, 92 are minus and plus inputs, respectively. Using certain techniques of this disclosure, clinician programmer 24 (or another device) may transmit parameters to patient programmer 26 during programming that assign functions to minus and plus inputs 90, 92. For example, for IMDs that deliver electrical stimulation therapy, minus and plus inputs 90, 92 may allow patient 6, 36 to decrease and increase values of neurostimulation parameter settings. In one implementation, buttons 90, 92 may permit patient 6, 36 to quickly decrease or increase the amplitude of stimulation being delivered by IMD 4, 34, for IMDs that deliver electrical stimulation therapy.

Or, for IMDs that deliver therapeutic agents, e.g., drug pumps, clinician programmer 24 may transmit parameters during programming of patient programmer 26 that assign functions to minus and plus inputs 90, 92 that decrease and increase the amount of the therapeutic agent(s) that are delivered by the IMDs. In some implementations, buttons 90, 92 may permit a patient to quickly decrease or increase the size of the bolus of the therapeutic agent that is delivered for IMDs that deliver therapeutic agents.

In this manner, minus and plus inputs 90, 92 may be initially associated with a generic functionality, e.g., decrease and increase. In accordance with one aspect of this disclosure, minus and plus inputs 90, 92 may then, via the parameters transmitted by clinician programmer 24 or another device, be assigned specific functions, e.g., decrease or increase the amplitude of stimulation being delivered for IMDs that deliver electrical stimulation therapy or decrease or increase the concentration of the therapeutic agent(s) that are delivered for IMDs that deliver therapeutic agents. In some implementations, the parameters are part of the user-selected application that is transmitted from clinician programmer 24 to patient programmer 26.

As indicated above, devices other than a clinician programmer may transmit the parameters to patient programmer 26. For example, a general-purpose personal computer (PC) (e.g., a desktop PC, a laptop PC, and the like), a tablet PC, a network server, or some other device via a wired or wireless connection may transmit parameters to patient programmer 26 that assign, or map, functions to the input mechanisms, e.g., buttons, scroll wheels, soft keys, and the like, of patient programmer 26. In addition, in some example implementations, the parameters may be transmitted during the manufacturing process, or prior to shipment to a clinician. In other example implementations, the parameters may be transmitted at the time of implant of an IMD, during the initial programming of the IMD in the patient, or when an external programmer previously programmed with a first application is to be reprogrammed, or re-imaged, with a second application that is different than the first application.

Although not depicted, patient programmer 26 may include a scroll wheel. The scroll wheel may be in place of or in addition to minus and plus buttons 90, 92. As one example, the scroll wheel may be assigned, via transmitted parameters from clinician programmer 24 or another device, similar functions, e.g., decrease or increase, as described above. In some example implementations, the scroll wheel may be a physical scroll wheel. In other implementations, the scroll wheel may be a scroll wheel displayed on display 88 that receives input via a touchscreen.

Button 94 may be an on/off button that turns power on and off, and which also functions to turn backlighting on and off. Button 102 is a four-way (up, down, left, right) rocker switch that permits navigation through items presented on display 88.

Buttons 100 may be devoted to a variety of functions that may be assigned, or mapped, to patient programmer 26 during programming by clinician programmer 24. Like with minus and plus inputs 90, 92, buttons 100 may be inputs that are initially associated with generic functionalities such as activation of therapy, deactivation of therapy, and interrogation of an IMD to check device status. In accordance with one aspect of this disclosure, inputs 100 may be assigned specific functions via the parameters transmitted by clinician programmer 24. For example, for IMDs that deliver electrical stimulation, inputs 100 may be assigned functions such as activation of stimulation, deactivation of stimulation, and interrogation of IMD 4, 34 to check device status. The device status may include remaining battery power and current stimulation parameter settings, and may be displayed on display 88. As another example, for IMDs that deliver therapeutic agents, e.g., drug pumps, inputs 100 may be assigned functions such as activation of delivery of a therapeutic agent, deactivation of delivery of a therapeutic agent, and interrogation of an IMD to check device status. The device status may include remaining amount of therapeutic agent(s) and current delivery parameter settings, such as the bolus size and frequency of bolus delivery, and may be displayed on display 88.

In addition, soft keys 106, 108 may be inputs presented on display 88 whose functions may also be assigned to patient programmer 26 during programming by clinician programmer 24. Of course, there may be other soft keys and, as mentioned above, one or more scroll wheels, displayed on display 88 that may be inputs whose functions are assigned via transmitted parameters.

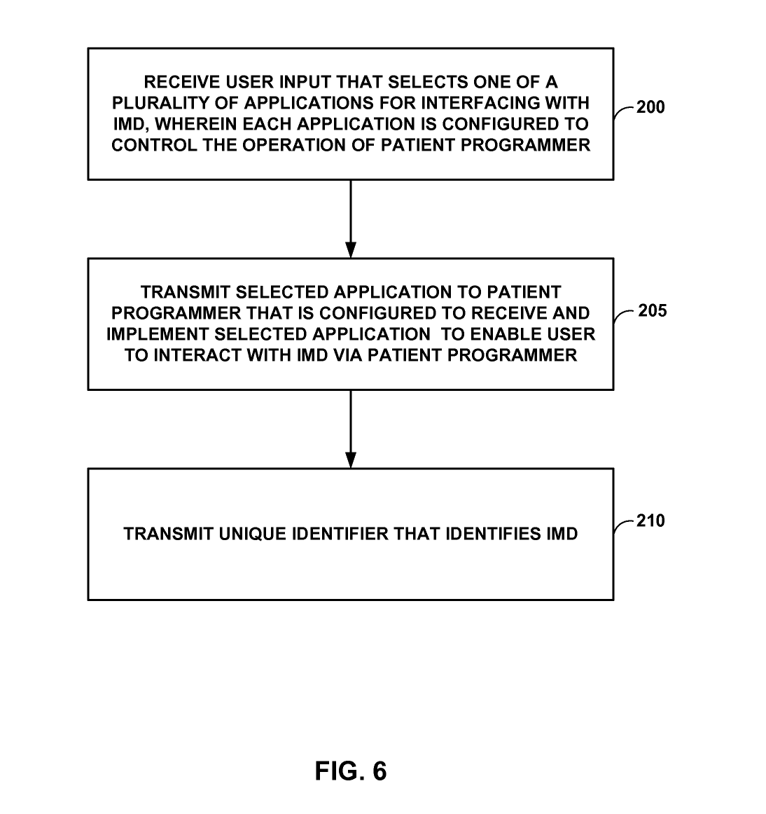

FIG. 6 is a flow diagram illustrating an example method for configuring a generic external programmer for use with implantable medical devices using certain techniques of this disclosure. In the example method shown in FIG. 6, a clinician programmer receives user input via a user interface that selects one of a plurality of applications for interfacing with the IMD, wherein each of the applications is configured to control the operation of the patient programmer (200). For example, at the time of initial programming of IMD 4 or IMD 34, such as at the time of the implant, or during a reprogramming of a previously programmed patient programmer, a clinician programmer 24 receives user input via user interface 42 that selects one of applications 54-56 as an application for interfacing with the IMD. Once the application is selected, processor 40 of clinician programmer 24 controls transmission of the selected application to a patient programmer, e.g., patient programmer 26, that is configured to receive and implement the application to enable a user to interact with the IMD via the patient programmer (205). The example method shown in FIG. 6 further shows the optional step of transmitting, from clinician programmer 24 to patient programmer 26, a unique identifier that identifies the IMD (210). In some example implementations, clinician programmer 24 transmits patient information to patient programmer 26, such as at least one of a patient name, a patient implant date, an implanting physician name, and a reason for implant.

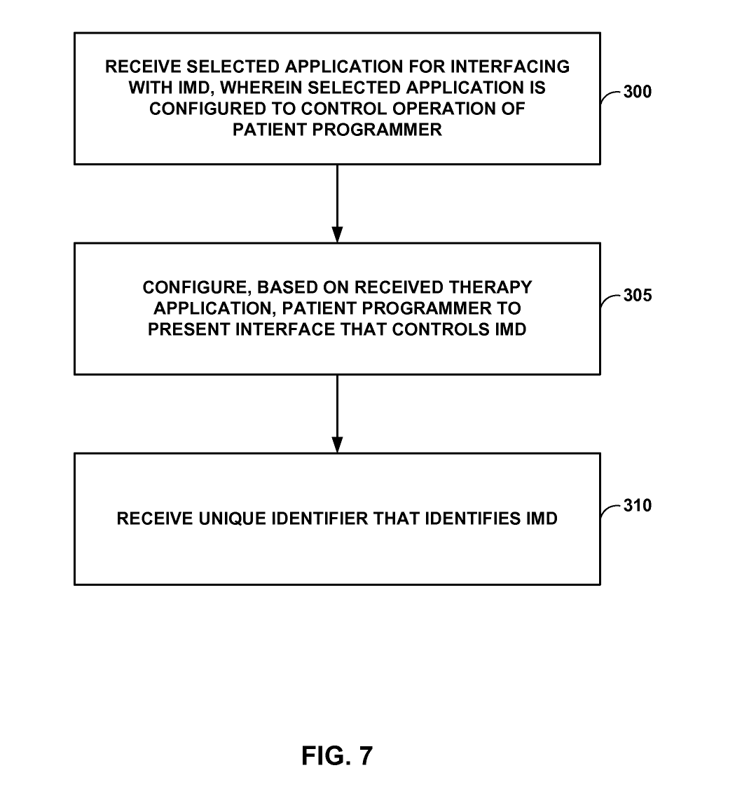

FIG. 7 is a flow diagram illustrating another example method for configuring a generic external programmer for use with implantable medical devices using certain techniques of this disclosure. In the example method shown in FIG. 7, a patient programmer receives a selected application for interfacing with the IMD, wherein the selected application is configured to control the operation of the patient programmer (300). For example, at the time of initial programming of IMD 4 or IMD 34, such as at the time of the implant, or during a reprogramming of a previously programmed patient programmer, patient programmer 26 receives from clinician programmer 24 a user-selected application 74 as an application for interfacing with the IMD. Once the selected application is received, processor 60 of patient programmer 26 configures the patient programmer to present an interface to a user that is specific to controlling the IMD based on the received application (305). The example method shown in FIG. 7 further shows the optional step of receiving, from clinician programmer 24, a unique identifier that identifies the IMD (310). In some example implementations, patient programmer 26 receives patient information from clinician programmer 24, such as at least one of a patient name, a patient implant date, an implanting physician name, and a reason for implant.

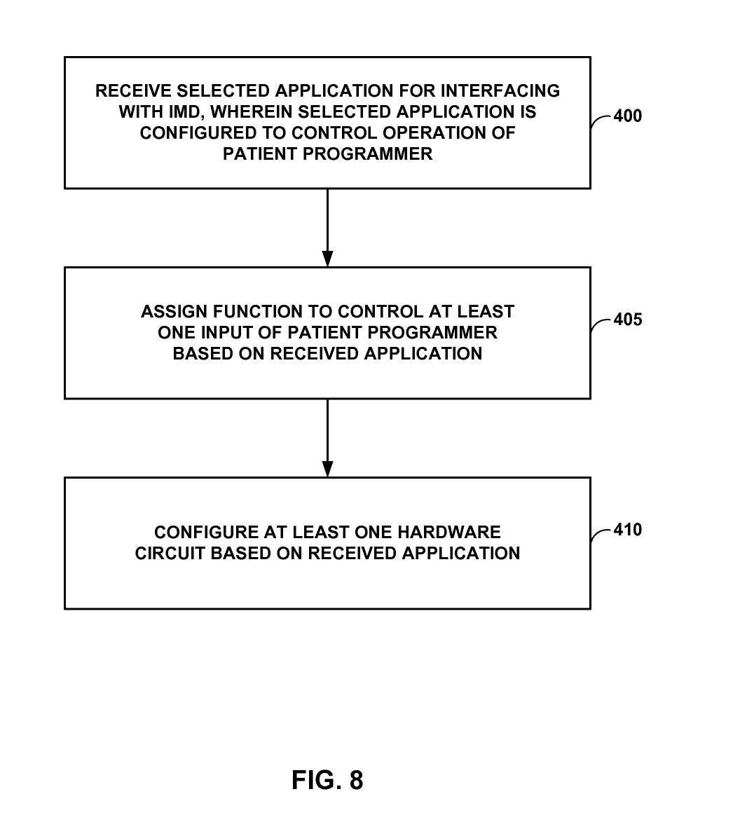

FIG. 8 is a flow diagram illustrating another example method for configuring a generic external programmer for use with implantable medical devices using certain techniques of this disclosure. In the example method shown in FIG. 8, a patient programmer receives a selected application for interfacing with the IMD from a clinician programmer, wherein the selected application is configured to control the operation of the patient programmer (400). For example, at the time of initial programming of IMD 4, IMD 34, or an IMD that delivers a therapeutic agent, such as at the time of the implant, or during a reprogramming of a previously programmed patient programmer, patient programmer 26 receives a user-selected application 74 (from amongst applications 54-56) as an application for interfacing with the IMD. Clinician programmer 24, a general-purpose personal computer (PC) (e.g., a desktop PC, a laptop PC, and the like), a tablet PC, a network server, or some other device via a wired or wireless connection may transmit the parameters received by patient programmer 26 that assign, or map, functions to the input mechanisms, e.g., buttons, scroll wheels, soft keys, and the like, of patient programmer 26. Once the selected application is received, processor 60 of patient programmer 26 assigns a function to control the IMD to at least one input of the patient programmer based on the received application and, in particular, parameters in the received application (405). For example, for IMDs that deliver electrical stimulation therapy, a device status input of the patient programmer may be configured to display remaining battery power, but for IMDs that deliver a therapeutic agent, a device status input of the patient programmer may be configured to display the remaining amount of the therapeutic agent. The example method shown in FIG. 8 further shows the optional step of configuring at least one hardware circuit based on the received application and, in particular, parameters in the received application (410). For example, the received application may configure the telemetry circuit, e.g., telemetry module 64, or the display of patient programmer 26.

As described above, the techniques of this disclosure allow a generic external programmer, e.g., a patient programmer, to be customized at the time of initial programming of an implantable medical device (IMD) or during a reprogramming of a previously programmed patient programmer, for example, with an application for interfacing with the implanted medical device. These techniques may provide a number of advantages, including simplifying the manufacturing process, supply chain management, inventory control, hardware distribution, and new product introductions.

Although the techniques discussed throughout this disclosure are described generally with respect to transmitting applications from a clinician programmer to a patient programmer, in some example implementations, other devices may be used to transmit applications to a patient programmer. For example, a general-purpose personal computer (PC) (e.g., a desktop PC, a laptop PC, and the like), a tablet PC, a network server, or some other device via a wired or wireless connection may transmit applications to a patient programmer using the techniques described above.

The techniques described in this disclosure may be implemented, at least in part, in hardware, software, firmware or any combination thereof. For example, various aspects of the techniques may be implemented within one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The terms “processor,” “processing circuitry,” “controller” or “control module” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as random access memory (RAM), read-only memory (ROM), non-volatile random access memory (NVRAM), electrically erasable programmable read-only memory (EEPROM), FLASH memory, magnetic media, optical media, or the like. The instructions may be executed to support one or more aspects of the functionality described in this disclosure.

Various aspects of the disclosure have been described. These and other aspects are within the scope of the following claims.

1. A method comprising:
receiving, via a clinician programmer, user input that selects one of a plurality of applications, wherein each of the applications is configured to control the operation of a patient programmer, the selected application is associated with a type of the IMD, and others of the plurality of applications are associated with different types of IMDs; and
transmitting, via the clinician programmer, the selected application to the patient programmer, wherein the patient programmer is configured to receive and implement the selected application to enable a user to interact with the IMD via the patient programmer.

2. The method of claim 1, wherein the selected application enables the user to control delivery of at least one of a therapeutic electrical stimulation and a therapeutic drug.

3. The method of claim 1, further comprising:
transmitting, from the clinician programmer to the patient programmer, a unique identifier that identifies the IMD.

4. The method of claim 1, further comprising:
transmitting, from the clinician programmer to the patient programmer, patient information, wherein the patient information comprises at least one of a patient name, a patient implant date, an implanting physician name, and a reason for implant.

5. The method of claim 1, wherein transmitting the selected application to a patient programmer is via a wired connection.

6. The method of claim 1, further comprising:
transmitting parameters that assign a function to at least one input of the patient programmer based on the selected application.

7. A system comprising:
a user interface;
a memory device configured to store a plurality of applications, wherein each of the applications is configured to the control the operation of a patient programmer; and
a processor configured to:
receive user input that selects one of the plurality of applications at the time of initial programming of the implantable medical device (IMD), wherein the selected application is associated with a type of the IMD, and others of the plurality of applications are associated with different types of IMDs; and
control transmission of the selected application to the patient programmer, wherein the patient programmer is configured to receive and implement the selected application to enable a user to interact with the IMD via the patient programmer.

8. The system of claim 7, wherein the selected application enables the user to control delivery of at least one of a therapeutic electrical stimulation and a therapeutic drug.

9. The system of claim 7, wherein the processor is further configured to:
control transmission of a unique identifier that identifies the IMD.

10. The system of claim 7, wherein the processor is further configured to:
control transmission of patient information, wherein the patient information comprises at least one of a patient name, a patient implant date, an implanting physician name, and a reason for implant.

11. The system of claim 7, wherein the processor is further configured to:
control transmission of the selected application via a wired connection.

12. The system of claim 7, wherein the processor is further configured to:
control transmission of parameters that assign a function to at least one input of the patient programmer based on the selected application.

13. A computer-readable storage medium comprising instructions that, when executed by a processor, cause the processor to:
receive user input that selects one of a plurality of applications at the time of initial programming of the implantable medical device (IMD), wherein the selected application is associated with a type of the IMD, and others of the plurality of applications are associated with different types of IMDs; and
control transmission of the selected application to the patient programmer, wherein the patient programmer is configured to receive and implement the selected application to enable a user to interact with the IMD via the patient programmer.

14. The computer-readable storage medium of claim 13, wherein the selected application enables the user to control delivery of at least one of a therapeutic electrical stimulation and a therapeutic drug.

15. The computer-readable storage medium of claim 13, further comprising instructions that, when executed by a processor, cause the processor to:
control transmission of a unique identifier that identifies the IMD.

16. The computer-readable storage medium of claim 13, further comprising instructions that, when executed by a processor, cause the processor to:
control transmission of patient information, wherein the patient information comprises at least one of a patient name, a patient implant date, an implanting physician name, and a reason for implant.

17. The computer-readable storage medium of claim 13, further comprising instructions that, when executed by a processor, cause the processor to:
control transmission of the selected application to a patient programmer via a wired connection.

18. The computer-readable storage medium of claim 13, further comprising instructions that, when executed by the processor, cause the processor to:
control transmission of parameters that assign a function to at least one input of the patient programmer based on the selected application.

19. A method comprising:
receiving, from a clinician programmer, a selected one of a plurality of applications, wherein each of the applications is configured to control the operation of a patient programmer, the selected application is associated with a type of the IMD, and others of the plurality of applications are associated with different types of IMDs; and
configuring, based on the received application, the patient programmer to present an interface that enables a user to interact with the IMD via the patient programmer.

20. The method of claim 19, wherein the selected application enables the user to control delivery of at least one of a therapeutic electrical stimulation and a therapeutic drug.

21. The method of claim 19, further comprising:
receiving a unique identifier that identifies the IMD.

22. The method of claim 19, further comprising:
receiving patient information, wherein the patient information comprises at least one of a patient name, a patient implant date, an implanting physician name, and a reason for implant.

23. The method of claim 19, wherein receiving the selected application is via a wired connection.

24. The method of claim 19, further comprising:
assigning, based on the received application, a function to at least one input of the patient programmer.

25. A system comprising:
a user interface;
a memory device configured to store an application, wherein the application is configured to control the operation of a patient programmer; and
a processor configured to:
receive, from a clinician programmer, a selected one of a plurality of applications for interfacing with an implantable medical device (IMD), wherein the selected application is associated with a type of IMD, and others of the plurality of applications are associated with different types of IMDs; and
configure, based on the received application, the user interface to present an interface that enables a user to interact with the IMD via the patient programmer.

26. The system of claim 25, wherein the selected application enables the user to control delivery of at least one of a therapeutic electrical stimulation and a therapeutic drug.

27. The system of claim 25, wherein the processor is further configured to:
receive a unique identifier that identifies the IMD.

28. The system of claim 25, wherein the processor is further configured to:
receive patient information, wherein the patient information comprises at least one of a patient name, a patient implant date, an implanting physician name, and a reason for implant.

29. The system of claim 25, wherein the processor is further configured to:
receive the selected application via a wired connection.

30. The system of claim 25, wherein the processor is further configured to:
assign, based on the received application, a function to at least one input of the patient programmer.

31. A computer-readable storage medium comprising instructions that, when executed by a processor, cause the processor to:
receive, from a clinician programmer, a selected one of a plurality of applications for interfacing with an implantable medical device (IMD), wherein the selected application is associated with a type of IMD, and others of the plurality of applications are associated with different types of IMDs; and
configure, based on the received application, a user interface to present an interface that enables a user to interact with the IMD via the patient programmer.

32. The computer-readable storage medium of claim 31, wherein the selected application enables the user to control delivery of at least one of a therapeutic electrical stimulation and a therapeutic drug.

33. The computer-readable storage medium of claim 31, further comprising instructions that, when executed by a processor, cause the processor to:
receive a unique identifier that identifies the IMD.

34. The computer-readable storage medium of claim 31, further comprising instructions that, when executed by a processor, cause the processor to:
receive patient information, wherein the patient information comprises at least one of a patient name, a patient implant date, an implanting physician name, and a reason for implant.

35. The computer-readable storage medium of claim 31, further comprising instructions that, when executed by a processor, cause the processor to:
receive the selected application via a wired connection.

36. The computer-readable storage medium of claim 31, further comprising instructions that, when executed by a processor, cause the processor to:
assign, based on the received application, a function to at least one input of the patient programmer.

37. A method comprising:
receiving a selected one of a plurality of applications, wherein each of the applications is configured to control the operation of a patient programmer, the selected application is associated with a type of the IMD, and others of the plurality of applications are associated with different types of IMDs; and
assigning, based on the received application, a function to at least one input of the patient programmer.

38. The method of claim 37, wherein the selected application enables a user to control delivery of at least one of a therapeutic electrical stimulation and a therapeutic drug.
